SEQUENCE LISTING (3 sequences)

SEQ ID NO: 1 - DNA, 106 bp, synthetic construct
  source 1..106: mol_type = other DNA
atgtctttgcagccgaggaggagctggtggaggctgacgaggcgggcagtgtgtatgcaggcatcctcagctacggggtgggcttcttcctgttcatcctggtggt

SEQ ID NO: 2 - DNA, 25 bp, synthetic construct
  source 1..25: mol_type = other DNA
agctcctcctcggctgcaaagacat

SEQ ID NO: 3 - DNA, 25 bp, synthetic construct
  source 1..25: mol_type = other DNA
accaccaggatgaacaggaagaagc

(19) United States
(12) Patent Application Publication
(10) Pub. No.: US 2023/0384298 A1
(43) Pub. Date: Nov. 30, 2023

(54) POLYMER PARTICLE CONTAINING MAGNETIC MATERIAL, MEDIUM FOR SENSORS, AND SENSOR DEVICE

(71) Applicant: TDK CORPORATION, Tokyo (JP)

(72) Inventors: Yusuke ARIAKE, Tokyo (JP); Kazuhiro YOSHIDOME, Tokyo (JP); Isao KANADA, Tokyo (JP)

(73) Assignee: TDK CORPORATION, Tokyo (JP)

(21) Appl. No.: 18/175,036

(22) Filed: Feb. 27, 2023

(30) Foreign Application Priority Data
Feb. 28, 2022 (JP) 2022-030071

(51) Int. Cl. G01N 33/543; G01N 27/74; H01F 1/20

(52) U.S. Cl. CPC G01N 33/54326; G01N 27/745; H01F 1/20

(57) ABSTRACT

A polymer particle containing magnetic material includes a core, an intermediate layer, and a polymer layer. The core includes magnetic fine particles having an average diameter of 5 nm or more and 30 nm or less. The intermediate layer is located outside the core and has a lower concentration of the magnetic fine particles than the core. The polymer layer covers the intermediate layer. A thickness of the intermediate layer is 5% or more and 60% or less of a radius of the polymer particle containing magnetic material.

The present application contains a Sequence Listing that has been submitted electronically and is hereby incorporated by reference herein in its entirety. The electronic Sequence Listing is named 223766 Sequence Listing.xml, which was created on Feb. 27, 2023 and is 3,609 bytes in size.

### BACKGROUND OF THE INVENTION

The present invention relates to a polymer particle containing magnetic material capable of being used, for example, in magnetic biosensors.

In the fields of biochemistry, medicine, and the like, a magnetic biosensor is known as a technique for detecting proteins, nucleic acids, cells, and the like in specimens. The magnetic biosensor is a method for detecting the existence and concentration of target substances in specimens by detecting the existence and number of magnetic particles located near the surface of the detection unit. The magnetic biosensor can detect the target substances with high sensitivity and has an advantage of avoiding the use of unstable compounds as conventional detection methods using optical systems.

The magnetic biosensors are required to have a high sensitivity for detection of very small amounts of target substances. The magnetic particles used to achieve this are required to have a high saturation magnetization, a resistance to sedimentation in the detection unit, and a high dispersion stability. If the magnetic particles settle in the detection unit, they become noise components during signal detection, resulting in a decrease in detection sensitivity.

Patent Document 1 below proposes a method for producing magnetic particles. In Patent Document 1, non-magnetic particles having a diameter equal to or less than half a diameter of magnetic mother particles are provided on the surfaces of magnetic mother particles, and they are coated with a polymer. Patent Document 2 proposes a polymer-coated ferromagnetic particle. In Patent Document 2, ferrite particles are coated with a polymer layer and a polyglycidyl methacrylate (pGMA) layer, and the weight ratio of ferromagnetic particles is more than 33% and less than 88%.

Conventional magnetic particles as described above are excellent in dispersion stability, but have a problem with decrease in detection sensitivity because the polymer coating layer is thick, and the distance between the detection unit and the magnetic mother particles (or ferromagnetic particles) increases. In addition, conventional magnetic particles as described above have a problem with decrease in sensor sensitivity because if the coating layer is thin, the ratio of magnetic material increases, the specific gravity increases, and sedimentation into the detection unit occurs and becomes noise.
Patent Document 1: JP5003867 (B2)
Patent Document 2: JP2018133467 (A)

### BRIEF SUMMARY OF THE INVENTION

The present invention has been achieved under such circumstances. It is an object of the invention to provide a polymer particle containing magnetic material capable of reducing noise and improving detection sensitivity, a medium for sensors containing the polymer particle, and a sensor device using the polymer particle containing magnetic material.

To achieve the above object, a polymer particle containing magnetic material according to the present invention comprises:
a core including magnetic fine particles having an average diameter of 5 nm or more and 30 nm or less;
an intermediate layer located outside the core and having a lower concentration of the magnetic fine particles than the core; and
a polymer layer covering the intermediate layer,

wherein a thickness of the intermediate layer is 5% or more and 60% or less of a radius of the polymer particle containing magnetic material.

The present inventors have newly found that the polymer particle containing magnetic material of the present invention can achieve high sensor sensitivity and background signal (noise) reduction at the same time. This is probably because the formation of the intermediate layer with a predetermined thickness while controlling the distribution of magnetic fine particles in the polymer particle containing magnetic material can exhibit sufficient magnetic properties for detection and prevent sedimentation of the polymer particles. This is also probably because when the average diameter of the magnetic fine particles contained in the core and the intermediate layer is a predetermined value (e.g., 5 nm or more and 30 nm or less), a high saturation magnetization and a low coercivity (superparamagnetism) are obtained and exhibit sufficient magnetic properties for detection, and it is possible to prevent sedimentation due to magnetic aggregation of polymer particles.

Preferably, a thickness of the intermediate layer is 10% or more and 42% or less of a radius of the polymer particle containing magnetic material. If the thickness of the intermediate layer is too small, the thickness of the polymer layer becomes relatively large, or the region of the core becomes relatively large. If the thickness of the polymer layer becomes large, the total amount of magnetic fine particles contained in the polymer particles tends to decrease, and the detection sensitivity tends to decrease. If the region of the core becomes relatively large, the specific gravity of the polymer particle tends to become large, and sedimentation tends to easily occur.

Preferably, a ratio (x1/x2) of a diameter (x1) of the magnetic fine particles to a diameter (x2) of the polymer particle containing magnetic material is 0.005 or more and 0.25 or less. In this range, it is easy to manufacture a polymer particle achieving high sensor sensitivity and background signal (noise) reduction at the same time.

Preferably, a polymer constituting the polymer layer contains an unpolymerized vinyl group. Instead, preferably, an intensity ratio of a peak in 1620 to 1640 cm⁻¹ to a peak in 1590 to 1610 cm⁻¹ in a FT-IR spectrum is 0.2 or more and 3.0 or less. For example, when the polymer contains an unpolymerized vinyl group, the dispersion in the aqueous solution (water medium) is favorable, aggregation and sedimentation are less likely to occur, and an increase in background signal can be prevented.

The polymer particle containing magnetic material may further comprise a portion capable of directly or indirectly binding with a target substance.

The polymer particle containing magnetic material may be contained in a medium for sensors used for magnetic biosensor devices or the like.

A sensor device may comprise a sensor unit for detecting a magnetism of the polymer particle containing magnetic material binding with a target substance.

In the fields of biochemistry, medicine, and the like, a magnetic biosensor is known as a technique for detecting proteins, nucleic acids, cells, and the like in specimens. The magnetic biosensor is a method for detecting the existence and concentration of target substances in specimens by detecting the existence and number of magnetic particles located near the surface of the detection unit. The magnetic biosensor can detect the target substances with high sensitivity and has an advantage of avoiding the use of unstable compounds as conventional detection methods using optical systems. The polymer particle containing magnetic material according to the present invention can favorably be used as a medium for sensors of the magnetic biosensor.

### BRIEF DESCRIPTION OF THE DRAWING(S)

FIG. 1A is a schematic view of a polymer particle containing magnetic material according to an embodiment of the present invention;

FIG. 1B is a photomicrograph of a polymer particle containing magnetic material according to an example of the present invention;

FIG. 1C is a TEM (HAADF) image of a polymer particle containing magnetic material according to another embodiment of the present invention;

FIG. 2 is a graph illustrating a concentration distribution (intensity distribution) of magnetic fine particles of a polymer particle containing magnetic material according to examples and comparative examples of the present invention;

FIG. 3 is a graph illustrating a FT-IR analysis result of magnetic fine particles of a polymer particle containing magnetic material according to examples and comparative examples of the present invention;

FIG. 4 is a schematic diagram of a sensor device according to an embodiment of the present invention;

FIG. 5A is a schematic view illustrating an application of a polymer particle containing magnetic material according to an embodiment of the present invention;

FIG. 5B is a schematic view illustrating the next step of FIG. 5A;

FIG. 5C is a schematic view illustrating the next step of FIG. 5B;

FIG. 5D is a schematic view illustrating the next step of FIG. 5C;

FIG. 5E is a schematic view illustrating the next step of FIG. 5D;

FIG. 6A is a graph illustrating an example of output of a sensor device using a polymer particle containing magnetic material according to an example of the present invention; and

FIG. 6B is a graph illustrating an example of output of a sensor device using a polymer particle containing magnetic material according to a comparative example of the present invention.

### DETAILED DESCRIPTION OF THE INVENTION

Hereinafter, the present invention is described based on an embodiment shown in the figures.

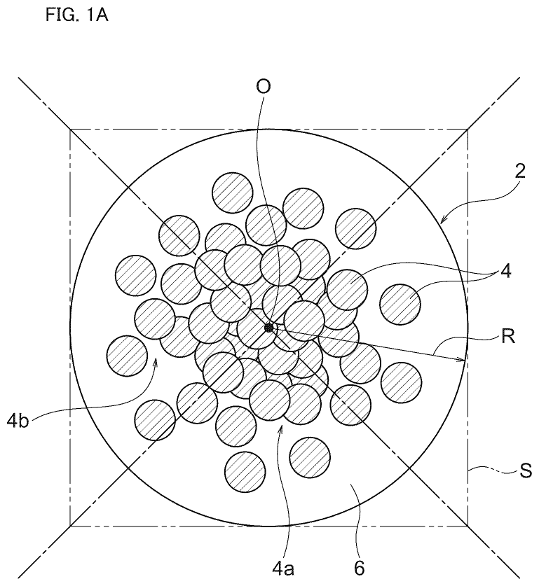

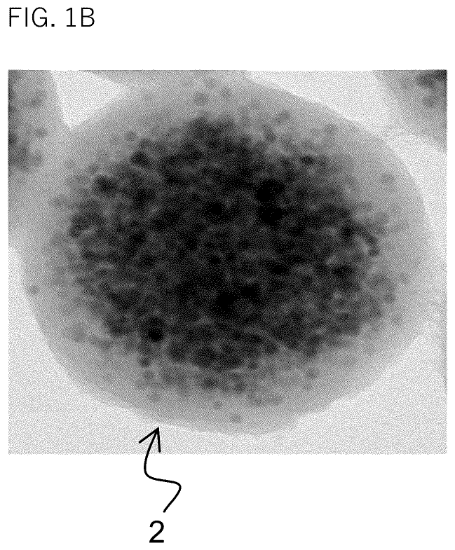

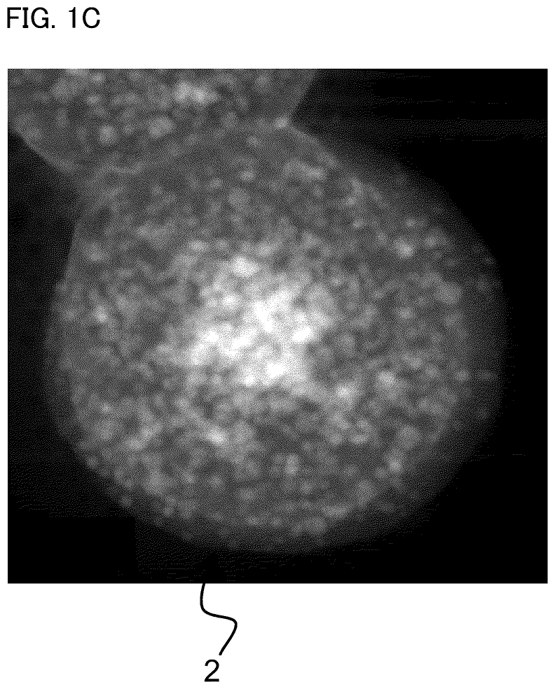

As shown in FIG. 1A and FIG. 1B, a polymer particle containing magnetic material (hereinafter, also referred to as a magnetic bead) 2 according to an embodiment of the present invention includes a core 4a containing magnetic fine particles 4 at a comparatively high concentration, an intermediate layer 4b containing the magnetic fine particles 4 at a lower concentration compared to the core 4a, and a polymer layer 6 covering the surface of the intermediate layer 4b.

The magnetic fine particles 4 are not limited as long as they are fine particles exhibiting ferromagnetism or superparamagnetism, but the magnetic fine particles 4 are preferably fine particles exhibiting superparamagnetism. For example, the magnetic material constituting the magnetic fine particles 4 is an iron oxide based compound, an iron nitride based compound, or the like, in addition to a single metal (e.g., Fe, Ni, and Co) and an alloy (e.g., a Fe—Ni alloy and a Fe—Co alloy). From the point of sufficient saturation magnetization and chemical stability, however, the magnetic material constituting the magnetic fine particles 4 is preferably an iron oxide based compound.

The iron oxide based compound includes a ferrite represented by MFe₂O₄(M=Co, Ni, Mg, Cu, Li_{0.5}Fe_{0.5}, etc.), a magnetite represented by Fe₃O₄, or γ-Fe₂O₃, but preferably includes either one of γ-Fe₂ O₃ and Fe₃ O₄ because of high saturation magnetization.

The magnetic fine particles 4 have an average diameter of 5 nm or more and 30 nm or less. The standard deviation σ indicating the dispersion of the diameters is preferably within 20% of the average diameter and is more preferably within 15% of the average diameter.

As shown in FIG. 1A, the intermediate layer 4b is defined as a region surrounding the core 4a and containing the magnetic fine particles 4 within a concentration range of 10% or more and 50% or less, compared to a highest concentration of the magnetic fine particles 4 inside the magnetic bead 2. The concentration of the magnetic fine particles 4 inside the magnetic bead 2 is determined, for example, as follows.

For example, as shown in FIG. 1C, a TEM (HAADF) image of the magnetic bead 2 is prepared.

From the photographed image shown in FIG. 1C (or FIG. 1B), as shown in FIG. 1A, a virtual quadrangle S circumscribing the outer contour of the magnetic bead 2 is determined, and an intersection point of the diagonal lines of the virtual quadrangle S is determined as a center O of the magnetic bead 2. Next, 12 virtual straight lines (not illustrated) are drawn every 30 degrees so as to divide the particle into 12 pieces from the center of the magnetic bead 2.

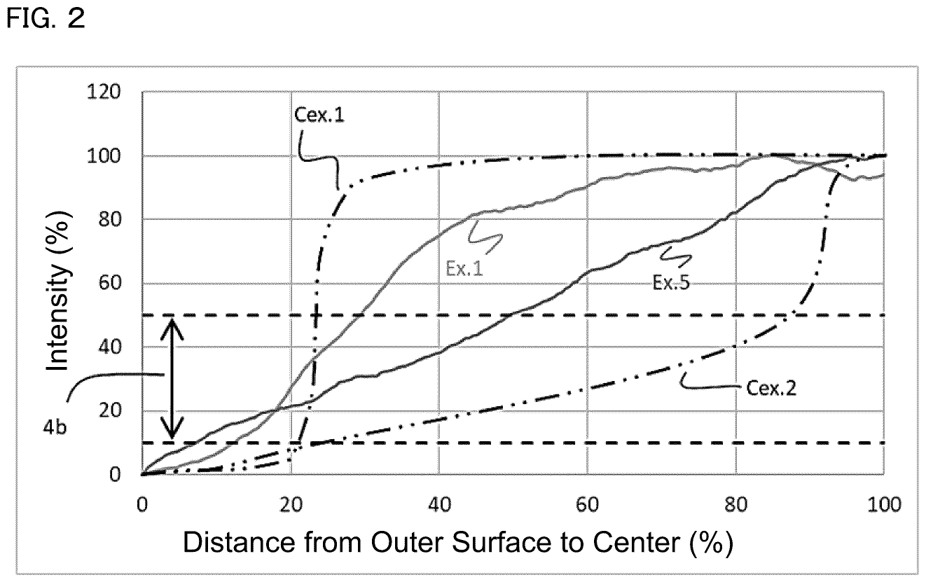

Next, a distance from the outer contour to the center O of the magnetic bead 2 is normalized from 0 to 100% along each of the virtual straight lines, and for example, a brightness intensity (corresponding to a concentration of the magnetic fine particles) of the image at each distance along each virtual straight line is obtained so as to calculate an average of the detected brightness intensities for the 12 virtual straight lines. The relation between the distance from the outer contour (outer surface) of the magnetic bead 2 and the detected brightness intensity (the concentration of the magnetic fine particles) obtained in such a manner can be obtained by average for a plurality (e.g., 10 or more) of magnetic beads 2 within an observation range. The detection intensity for brightness is normalized to 100 for the maximum value and 0 for the minimum value of the outer contour (polymer layer). FIG. 2 illustrates a graphed example of the relation between the distance from the outer surface of the magnetic bead 2 and the normalized detection intensity for brightness (corresponding to the concentration of the magnetic fine particles) obtained in such a manner.

In FIG. 2, the horizontal axis indicates a distance (%) from the outer contour (outer surface) of the magnetic bead 2 to the center, and the vertical axis indicates a detection intensity (%/corresponding to a concentration of the magnetic fine particles). The distance of 100% corresponds to a radius R of the magnetic bead 2 (see FIG. 1A). In the present embodiment, as shown in FIG. 2, the intermediate layer 4b is defined as a region where the magnetic fine particles 4 exist within a concentration (detection intensity) of 10% or more and 50% or less, compared to a portion where the concentration (detection intensity) of the magnetic fine particles 4 is highest (100%) inside the magnetic bead 2.

In FIG. 2, for example, the graph of Ex. 1 or Ex. 5 is obtained in the magnetic bead 2 within the scope of the embodiment of the present invention, and the graph of the curve Cex. 1 or Cex. 2 is obtained in a magnetic bead according to a comparative example. As shown in FIG. 2, the thickness of the intermediate layer 4b in the magnetic bead 2 within the scope of the embodiment of the present invention is about 16.5% and 41.5% as shown by the curves Ex. 1 and Ex. 5, respectively, which are within the scope (5% or more and 60% or less, preferably 10% or more and 42% or less) of the embodiment of the present invention.

A ratio (x1/x2) of a diameter (x1) of the magnetic fine particles 4 to a diameter (x2=2×R) of the magnetic bead 2 is preferably 0.005 or more and 0.25 or less, more preferably 0.01 or more and 0.2 or less, particularly preferably 0.01 or more and 0.15 or less. The diameter of the magnetic bead 2 is obtained as a circle equivalent diameter, for example, by performing an image analysis of the outer contour of the magnetic bead 2 from the observed image shown in FIG. 1B or FIG. 1C. The diameters of the magnetic fine particles 4 can be obtained in a similar manner.

In the polymer layer 6 of the magnetic bead 2, preferably, a polymer constituting the polymer layer 6 contains an unpolymerized vinyl group. Preferably, a monomer for forming the polymer layer 6 includes 50% by weight or more of a hydrophobic monomer. Here, the hydrophobic monomer is a single substance or a mixture of a polymerizable monomer whose solubility in water at 25° C. is 2.5% by weight or less. The hydrophobic monomer may be any of a monofunctional (non-crosslinkable) monomer and a crosslinkable monomer and may be a mixture of a monofunctional monomer and a crosslinkable monomer.

As a monofunctional monomer of the hydrophobic monomer, it is possible to exemplify an aromatic vinyl monomer, such as styrene, α-methylstyrene, and halogenated styrene, an ethylenically unsaturated carboxylic acid alkyl ester, such as methyl acrylate, ethyl acrylate, ethyl methacrylate, stearyl acrylate, stearyl methacrylate, cyclohexyl acrylate, cyclohexyl methacrylate, isobornyl acrylate, and isobornyl methacrylate, and the like. As a crosslinkable monomer of the hydrophobic monomer, it is possible to exemplify a polyfunctional (meth)acrylate, such as ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate, a conjugated diolefin, such as butadiene and isoprene, divinylbenzene, diallyl phthalate, allyl acrylate, allyl methacrylate, and the like.

The monomer constituting the polymer layer 6 may include a non-hydrophobic monomer (hydrophilic monomer). As a monofunctional monomer of the non-hydrophobic monomer, it is possible to exemplify a monomer having a carboxyl group, such as acrylic acid, methacrylic acid, maleic acid, and itaconic acid, an acrylate having a hydrophilic functional group (e.g., hydroxyl group, amino group, alkoxy group), such as 2-hydroxyethyl acrylate, 2-hydroxyethyl methacrylate, glycerol acrylate, glycerol methacrylate, methoxyethyl acrylate, methoxyethyl methacrylate, polyethylene glycol acrylate, polyethylene glycol methacrylate, 2-dimethylaminoethyl(meth)acrylate, 2-diethyl aminoethyl (meth)acrylate, 2-dimethyl aminopropyl (meth)acrylate, and 3-dimethylaminopropyl(meth)acrylate, acrylamide, methacrylamide, N-methylol acrylamide, N-methylol methacrylamide, diacetone acrylamide, N-(2-diethyl aminoethyl)(meth)acrylamide, N-(2-dimethyl aminopropyl)(meth)acrylamide, N-(3-dimethylaminopropyl)(meth)acrylamide, styrenesulfonic acid and its sodium salt, 2-acrylamido-2-methylpropanesulfonic acid and its sodium salt, isoprenesulfonic acid and its sodium salt, N,N-dimethylaminopropyl acrylamide and its methyl chloride quaternary salt, a copolymer with a copolymerizable monomer such as allylamine, and the like. As a cross-linkable monomer of the non-hydrophobic monomer, it is possible to exemplify a hydrophilic monomer, such as polyethylene glycol diacrylate, polyethylene glycol dimethacrylate, and poly(meth)acrylic ester of polyvinyl alcohol.

The polymer constituting the polymer layer 6 and the polymer dispersing the magnetic fine particles existing in the intermediate layer 4b are preferably the same continuous polymer, but they do not necessarily have to be the same polymer. Moreover, the polymer dispersing the magnetic fine particles 4 existing in the intermediate layer 4b and the polymer located among the magnetic fine particles 4 existing in the core 4a may be the same continuous polymer, but may be different polymers.

The polymer layer 6 may not contain the magnetic fine particles 4 at all, but may contain the magnetic fine particles 4. For example, the polymer layer 6 is defined as a region surrounding the intermediate layer 4b defined as described above and containing the magnetic fine particles 4 within a concentration of less than 10% (preferably 5% or less, and more preferably 3% or less including 0), compared to a portion where the concentration of the magnetic fine particles 4 is highest inside the magnetic bead 2.

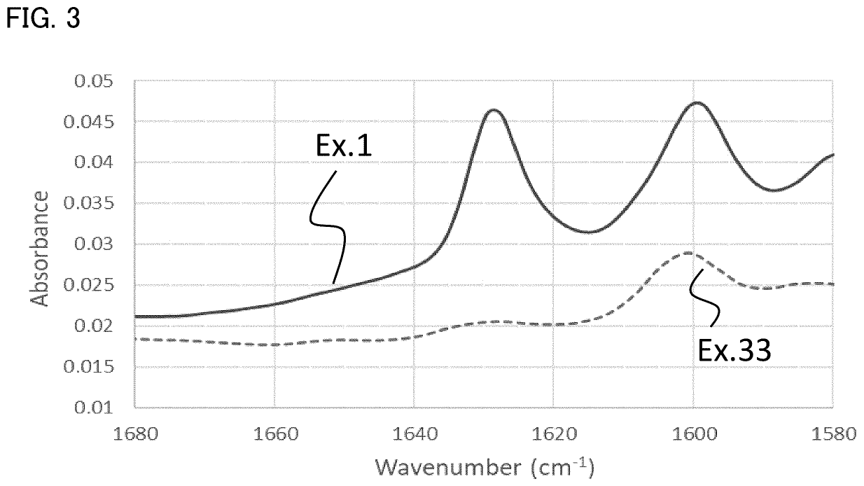

In the polymer layer 6 of the magnetic bead 2, as shown by the curve of Ex. 1 shown in FIG. 3, an intensity ratio of a peak in 1620 to 1640 cm⁻¹ to a peak in 1590 to 1610 cm⁻¹ in a FT-IR spectrum is preferably 0.2 or more and 3.0 or less and is more preferably 0.5 or more and 3.0 or less. The peak appearing in 1590 to 1610 cm⁻¹ is a peak derived from an aromatic ring, and the peak in 1620 to 1640 cm⁻¹ is a peak derived from an unpolymerized vinyl group.

In the determination of the peak intensity ratio, each peak intensity is defined as a height of the peak from a background straight line. The background straight line is a straight line connecting the points of 1590 cm⁻¹ and 1615 cm⁻¹ on the spectral curve for the peak in 1590 to 1610 cm⁻¹ and is a straight line connecting the points of 1615 cm⁻¹ and 1643 cm⁻¹ on the spectral curve for the peak in 1620 to 1640 cm⁻¹.

The polymer layer 6 of the magnetic bead 2 of the present embodiment may be added with a portion capable of directly or indirectly binding with a target substance to be detected. Instead, the outer surface of the polymer layer 6 may be provided with another polymer layer or a non-polymer layer added with a portion capable of directly or indirectly binding with a target substance to be detected.

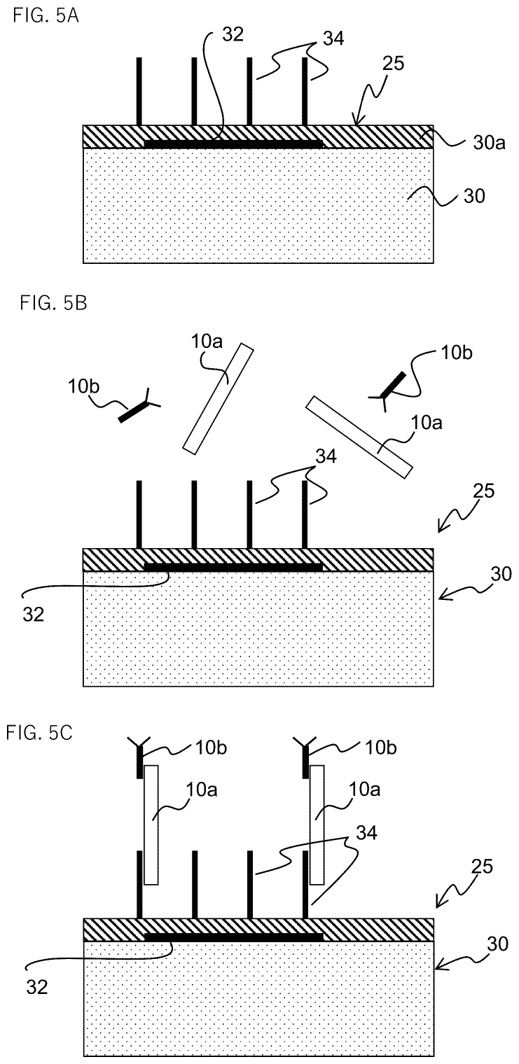

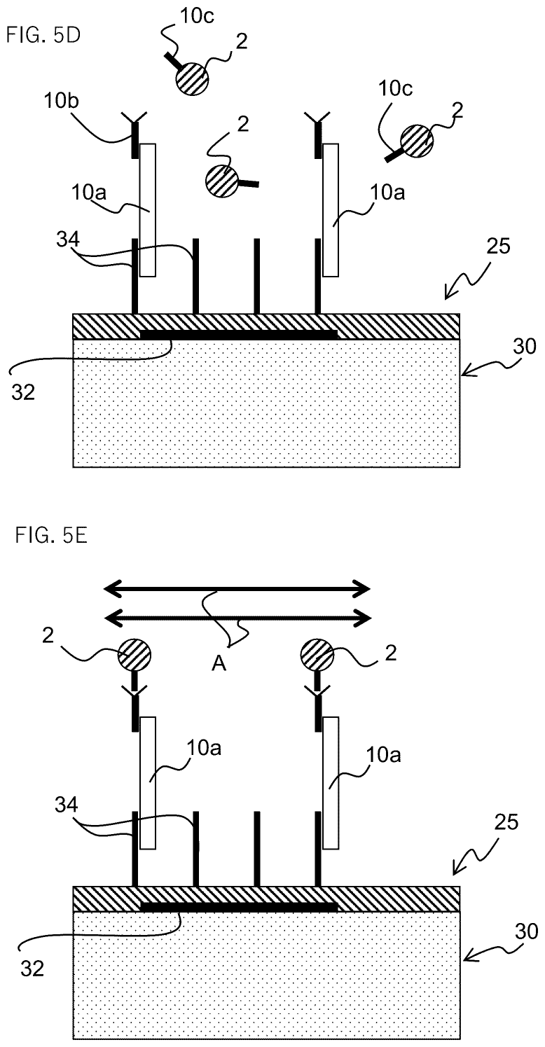

The target substance to be detected is not limited and is exemplified by, for example, a predetermined single-stranded nucleic acid 10a shown in FIG. 5D. The polymeric layer 6 of the magnetic bead 2 shown in FIG. 1A or another polymer layer or a non-polymer layer covering its surface may be capable of directly binding with the single-stranded nucleic acid 10a of the target substance shown in FIG. 5D or may be capable of binding with a binding auxiliary substance 10b or 10c being easy to bind with the single-stranded nucleic acid 10a of the target substance.

The magnetic bead 2 and the nucleic acid 10a as an example of the target substance are bound by any known method, such as coordinate bond, covalent bond, hydrogen bond, hydrophobic interaction, physical adsorption, and affinity bond, and may be bound indirectly via linkers or the like. As a specific binding method, for example, a functional group existing on the surface of the polymer layer 6 of the magnetic bead 2 and the nucleic acid 10a are bound by covalent bond. Moreover, there is a binding by interaction between the magnetic bead 2 added with the binding auxiliary substance 10c, such as avidin and streptavidin, and the nucleic acid having biotin or the binding auxiliary substance 10b.

Other examples of the binding auxiliary substance 10c as one linker include antibodies, antigens, protein A, and protein G. Other examples of the binding auxiliary substance 10b as the other linker include corresponding antigens or antibodies.

Next, a method for manufacturing the magnetic bead 2 according to present embodiment shown in FIG. 1A is described.

First, magnetic fine particles 4 having an average diameter of 5 nm or more and 30 nm or less are manufactured. The method for manufacturing the magnetic fine particles 4 is not limited and is, for example, coprecipitation method, thermal decomposition method, polyol method, sol-gel method, laser ablation method, thermal plasma method, and spray pyrolysis method.

Next, as shown in FIG. 1A, the magnetic fine particles 4 obtained in such a manner are dispersed or aggregated in a polymer at a comparatively high concentration to form a core 4a, and an intermediate layer 4b is formed around the core 4a, and a polymer layer 6 is further formed around the intermediate layer 4b to manufacture the magnetic bead 2.

In the present embodiment, first, the core 4a is manufactured. The core 4a is manufactured by any method and is manufactured, for example, as follows. First, the magnetic fine particles 4 are uniformly dispersed in a hydrophobic organic solvent, added into an aqueous solution in which a surfactant is dissolved, and emulsified to prepare a magnetic fine particle emulsion. The core 4a is formed by adding a monomer and a polymerization initiator thereto and causing a polymerization reaction.

Next, the intermediate layer 4b is formed around the core 4a. The intermediate layer 4b is formed by any method and is formed, for example, as follows. The intermediate layer 4b is formed by uniformly dispersing the magnetic fine particles 4 in a monomer so as to have a predetermined concentration, adding the dispersion to the core 4a together with a surfactant and a polymerization initiator, and causing a polymerization reaction.

After that, the polymer layer 6 is formed around the intermediate layer 4b. The polymer layer 6 is formed by any method and is formed, for example, as follows. The polymer layer 6 is formed by adding a predetermined amount of a monomer together with a surfactant and a polymerization initiator to the particles formed from the core 4a and the intermediate layer 4b and causing a polymerization reaction.

When the polymer constituting the polymer layer 6 and the polymer for dispersing the magnetic fine particles 4 in the intermediate layer 4b are the same type of polymer, the polymer layer 6 and the intermediate layer 4b can be formed at the same time by the method as mentioned above. When all of the polymer for densely gathering the magnetic fine particles 4 in the core 4a, the polymer in the intermediate layer 4b, and the polymer in the polymer layer 6 are the same type of polymer, the core 4a, the intermediate layer 4b, and the polymer layer 6 can also be formed at the same time.

Next, a specific method of using the magnetic bead (polymer particle containing magnetic material) 2 according to the present embodiment is described.

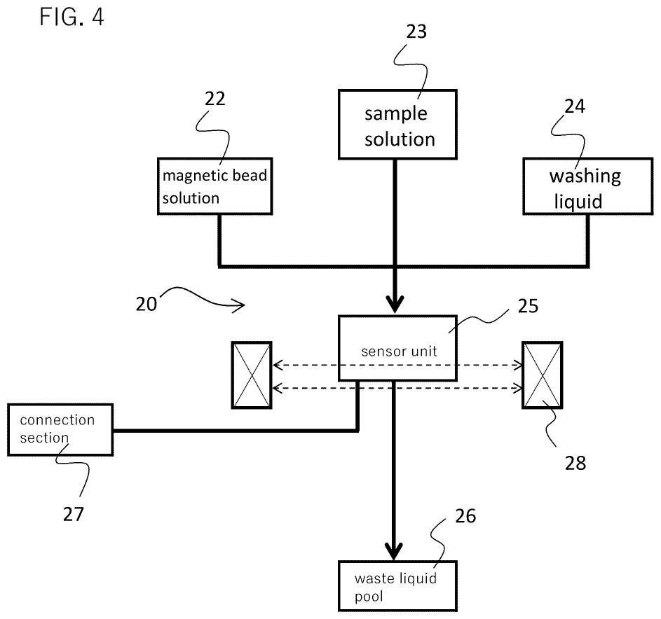

For example, a large number of magnetic beads 2 shown in FIG. 1A are dispersed in an aqueous solution, stored or transported as a magnetic bead solution, and stored in a magnetic bead storage 22 of a nucleic acid detection cartridge 20 shown in FIG. 4 used as, for example, a part of a sensor device. For example, a phosphate-buffered saline is used as the liquid for dispersing the magnetic beads 2. The cartridge 20 is used for sensing the existence or amount of a specific single-stranded nucleic acid 10a shown in FIG. 5B to FIG. 5E in a sample solution stored in a sample solution storage 23.

In the present embodiment, the cartridge 20 may include a washing liquid storage 24 and a waste liquid storage 26, in addition to the magnetic bead storage 22 and the sample solution storage 23. A washing liquid is stored in the washing liquid storage 24. The washing liquid, the sample solution, the bead solution, or the like that is no longer needed in a sensor unit 25 is discharged to the waste liquid storage 26. The washing liquid is, for example, a phosphate-buffered saline.

The cartridge 20 also includes the sensor unit 25, and the sensor unit 25 is connected to a connection section 27 for transmitting and receiving signals to and from an external circuit. The connection section 27 may be an electrical connection section or may be a connection section for optical or wireless communication. A magnetic field application unit 28 is attached to either the cartridge 20 or a device for attaching the cartridge 20. The magnetic field application unit 28 applies, for example, a magnetic field as shown by the arrows A in FIG. 5E to the magnetic beads 2 bound to the single-stranded nucleic acid 10a as a target substance. Note that, the application direction of the magnetic field shown in FIG. 5E is an example for description, and the application direction of the magnetic field is not limited to the arrows A.

As shown in FIG. 5A, for example, the sensor unit 25 shown in FIG. 4 includes at least one type of sensor element 32 inside a substrate 30 with a protection film 30a. Capture probes 34 are arranged on the surface of the substrate 30 (the surface of the protection film 30a) located above the sensor element 32. Preferably, for example, the capture probes 34 contain a nucleic acid having a sequence complementary to at least a part of a double-stranded nucleic acid or a single-stranded nucleic acid as a target substance, but there is no limitation as long as it is a substance capable of capturing a target substance.

The capture probes 34 may be composed of DNA, RNA, or a combination of them. From the point of preventing degradation by DNA degrading enzymes and RNA degrading enzymes, the capture probes 34 may contain an artificial nucleic acid.

Examples of methods for immobilizing the capture probes 34 onto the substrate 30 include a method using photolithography and solid-phase chemical reaction, a method of dropping a solution containing a capture probe onto the substrate for immobilization, and the like. In the method using photolithography and solid-phase chemical reaction, each of the capture probes 34 may be synthesized on the substrate 30.

In the method of dropping a solution containing the capture probes 34 onto the substrate for immobilization, preferably, a functional group for immobilization onto the substrate (hereinafter, sometimes abbreviated as “immobilization group”) is provided at the ends of the capture probes 34, and a functional group capable of reacting with the immobilization group and forming a bond (hereinafter, sometimes abbreviated as “reactive group”) is also formed on the substrate. Examples of the combinations between the immobilization group and reactive group include a combination between an immobilization group, such as amino group, formyl group, thiol group, and succimidyl ester group, and a reactive group, such as carboxyl group, amino group, formyl group, epoxy group, and maleimide group, a combination using a gold-thiol bond, and the like.

Examples of other methods of dropping a solution containing the capture probes 34 onto the substrate 30 for immobilization include a method of discharging the capture probes 34 having a silanol group at the ends onto a substrate having a silica portion on the sensor element, arranging them, and covalently bonding them by a silane coupling reaction.

Preferably, the sensor element 32 is a magnetic sensor element. This is because the detection signal increases according to the number of magnetic beads 2, and the concentration of the single-stranded nucleic acid (or double-stranded nucleic acid) as a target substance can be quantified with a high accuracy. For example, the magnetic sensor element can be a magnetoresistive element. The magnetoresistive effect element is not limited as long as it is an element utilizing a phenomenon in which the electric resistance value changes under the influence of a magnetic field, but is preferably an element provided with a magnetization fixed layer having a magnetization direction fixed in a predetermined direction in the lamination plane and a magnetization free layer whose magnetization direction changes according to an external magnetic field.

In the magnetoresistive element, the magnetization fixed direction of the magnetization fixed layer is substantially parallel or substantially antiparallel to the magnetic field applied for excitation of the magnetic beads and is the film surface direction of the magnetoresistive element. Note that, “substantially parallel” may be approximately parallel and may be deviated within a range of 10° or less.

Note that, the magnetoresistive element may be a giant magnetoresistive element (GMR element), a tunnel magnetoresistive element (TMR element), or the like, and that the electrical resistance value of the magnetoresistive element may change according to an angle between a magnetization direction of the magnetization fixed layer and an average magnetization direction of the magnetization free layer. The shape of the magnetoresistive element is not limited, but preferably has a meandering structure.

The magnetization free layer is composed of, for example, a soft magnetic film of a NiFe alloy or the like. One surface of the magnetization fixed layer is in contact with an antiferromagnetic film, and the other surface of the magnetization fixed layer is in contact with the intermediate layer. The antiferromagnetic film is composed of, for example, an antiferromagnetic Mn alloy, such as IrMn and PtMn. The magnetization fixed layer may be composed of a ferromagnetic material, such as a CoFe alloy and a NiFe alloy, or may have a structure in which a Ru thin film layer is sandwiched between ferromagnetic materials, such as a CoFe alloy and a NiFe alloy.

The sensor element 32 in FIG. 5A to FIG. 5E is disposed inside the substrate 30, but may be disposed on the surface of the substrate depending on the type of sensor element 32. A single type of sensor element 32 is exemplified as the sensor element 32, but a plurality of types of sensor elements 32 may be arranged inside or on the surface of the substrate 30 depending on the purpose.

The sample solution storage 23 shown in FIG. 4 stores a sample solution containing, for example, a double-stranded nucleic acid or the single-stranded nucleic acid 10a shown in FIG. 5B as a target substance and the binding auxiliary substance 10b. When the sample solution enters the sensor unit 25 from the sample solution storage 23, as shown in FIG. 5B and FIG. 5C, the sample solution comes into contact with the sensor unit 25, and the single-stranded nucleic acid 10a is captured by the capture probes 34. Before or after that, the single-stranded nucleic acid 10a and the binding auxiliary substance 10b also bind with each other. The single-stranded nucleic acid 10a and the binding aid substance 10b may be bound in advance.

When a free single-stranded nucleic acid 10a exists in the subsequent measurement system, the measurement accuracy decreases. Thus, after the single-stranded nucleic acid 10a and the capture probes 34 are bound, the free single-stranded nucleic acid is removed from the sensor unit 25 by, for example, a washing step of flowing a washing solution from the washing liquid storage 24 to the sensor unit 25 shown in FIG. 4 and washing it away to the waste liquid storage 26.

Next, when the magnetic bead solution is supplied from the magnetic bead storage 22 shown in FIG. 4 to the sensor unit 25, as shown in FIG. 5D and FIG. 5E, the auxiliary binding substance 10c of the magnetic bead 2 binds to the auxiliary binding substance 10b bound to the single-stranded nucleic acid 10a captured by the capture probes 34 in the sensor unit 25.

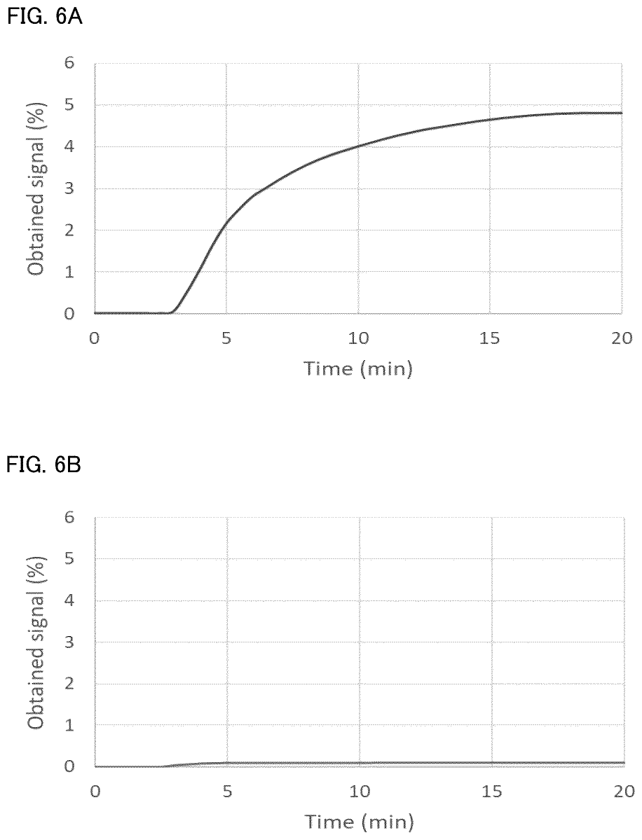

A magnetic field A is applied toward the captured magnetic bead 2 shown in FIG. 5E by the magnetic field application unit 28 concurrently with or before the supply of the magnetic bead solution from the magnetic bead storage 22 to the sensor unit 25 shown in FIG. 4. A change in the magnetic field from the magnetic bead 2 excited by the magnetic field A is detected as a change in resistance by the sensor element 32. FIG. 6A shows an example of the detection result.

In FIG. 6A, the horizontal axis represents an elapsed measurement time, and the vertical axis represents an output of the signal detected by the sensor element 32. The output of the sensor element 32 is continuously measured and, for example, these output saturation values can be used so as to obtain a concentration of the target single-stranded nucleic acid calculated from the output of the sensor element 32. The calculation of the concentration of the target single-stranded nucleic acid can be determined in advance by a nucleic acid detector (not illustrated) so that it can be automatically calculated from the measurement results. The process in which the magnetic beads 2 are adsorbed to the capture probes 34 on the sensor element 32 can be measured in real time with the nucleic acid detection cartridge 20 of the present embodiment.

According to the nucleic acid detection cartridge 20 of the present embodiment, it is possible to detect a target substance with high sensitivity, and there is an advantage that it is not necessary to use an unstable compound as in detection methods using conventional optical systems. The magnetic beads 2 of the present embodiment can be favorably used as a sensor medium for such a magnetic biosensor.

According to the magnetic beads 2 of the present embodiment, it is possible to achieve high sensor sensitivity and background signal (noise) reduction at the same time. This is probably because when the intermediate layer 6 having a predetermined thickness is formed by controlling the distribution of the magnetic fine particles in the polymer particle containing magnetic material, for example, the magnetic beads 2 can exhibit sufficient magnetic characteristics for detection by the sensor element 32 shown in FIG. 5E, and it is possible to prevent sedimentation of the magnetic beads 2. This is also probably because when the magnetic fine particles 4 contained in the core 4a and the intermediate layer 4b shown in FIG. 1A have an average diameter equal to or less than a predetermined value (e.g., 30 nm), a low coercivity (superparamagnetism) is obtained, and it is possible to prevent sedimentation due to mutual magnetic aggregation of the magnetic beads 2.

In the present embodiment, the thickness of the intermediate layer 4b shown in FIG. 1A and FIG. 2 is controlled at a predetermined ratio with respect to the particle radius of the magnetic beads 2. If the thickness of the intermediate layer 4 is too small, the thickness of the polymer layer 6 relatively becomes large, or the region of the core 4a relatively becomes large. If the thickness of the polymer layer 6 increases, the total amount of the magnetic fine particles 4 contained in the magnetic beads 2 tends to decrease, and the detection sensitivity tends to decrease. If the region of the core 4a relatively becomes large, the specific gravity of the magnetic beads 2 becomes large, and they tend to settle easily. The specific gravity of the magnetic beads 2 depends on the type of liquid for dispersing the magnetic beads 2 and is preferably 1.1 g/cm 3 or more and 2.6 g/cm 3 or less.

In the present embodiment, preferably, a ratio (x1/x2) of a diameter (x1) of the magnetic fine particles 4 to a diameter (x2) of the magnetic beads is 0.005 or more and 0.25 or less. In such a range, it is easy to manufacture magnetic beads achieving high sensor sensitivity and background signal (noise) reduction at the same time.

In the present embodiment, the polymer constituting the polymer layer 6 contains an unpolymerized vinyl group. Moreover, as shown in FIG. 3, an intensity ratio of a peak in 1620 to 1640 cm⁻¹ to a peak in 1590 to 1610 cm⁻¹ in a FT-IR spectrum of the magnetic beads 2 is preferably 0.2 or more and 3.0 or less and is more preferably 0.5 or more and 3.0 or less. For example, when the polymer contains an unpolymerized vinyl group, the magnetic beads 2 are favorably dispersed in an aqueous solution (aqueous medium), aggregation and sedimentation are less likely to occur, and it is possible to prevent an increase in background signal. When an intensity ratio of a peak in 1620 to 1640 cm⁻¹ to a peak in 1590 to 1610 cm⁻¹ is 0.2 or more, the effect of improvement in dispersibility is enhanced. When an intensity ratio of a peak in 1620 to 1640 cm⁻¹ to a peak in 1590 to 1610 cm⁻¹ is 3.0 or less, the structure of the particles becomes firm, and it is possible to stably detect the target substance.

The present invention is not limited to the above-mentioned embodiment and may be changed variously within the scope of the present invention.

For example, the sensor device using the magnetic bead 2 as the polymer particle containing magnetic material according to the present embodiment is not limited to the nucleic acid detection cartridge 20 shown in FIG. 4 and can be various sensor devices. Moreover, the target substance of the sensor device is not limited to a double-stranded nucleic acid or a single-stranded nucleic acid and may be other substances capable of binding to the polymer particle containing magnetic material.

### EXAMPLES

Hereinafter, the present invention is described based on more detailed examples, but is not limited to them.

### Example 1

### <Producing Magnetic Fine Particles and Magnetic Beads>

An iron (III) chloride hexahydrate was mixed with an ion-exchanged water and an ethanol and stirred for 30 minutes with a mechanical stirrer. After that, a solvent (hexane) was poured in, a sodium oleate was added, and the mixture was further stirred for 30 minutes. The solution was heated with stirring until the solution temperature reached about 59° C. and maintained at that temperature for 4 hours to synthesize an iron oleate. After cooling the solution, the solution was recovered, and an aqueous layer and an oil layer were separated with a separatory funnel so as to recover the oil layer.

A washing was performed by adding an ion-exchanged water to the oil layer and stirring it so as to remove the water layer. After this washing was repeated three times, the oil layer was recovered. A hexane solution of the obtained iron oleate was purified (removal of hexane) using an evaporator or the like so as to obtain an iron oleate (a waxy liquid with high viscosity).

The iron oleate as a raw material was stirred together with a dispersant (oleic acid) in a solvent (octadecene) at 120° C. for 2 hours for dissolution. After that, the temperature of the solution was increased, and the solution was subjected to a thermal decomposition for 2 hours at 317° C. (boiling point) while being refluxed. The solution after cooling was added with an ethanol for washing and stirred, and iron oxide nanoparticles (magnetic fine particles) were thereafter recovered by performing a centrifugation and removing the supernatant. This was repeated 5 times.

The obtained iron oxide nanoparticles were dispersed in octane to produce magnetic beads 2 as shown in FIG. 1A. Specifically, the magnetic beads 2 were produced as follows. That is, first, magnetic fine particles 4 were uniformly dispersed in n-octane so that the concentration would be 50 wt %, and a magnetic fine particle dispersion was prepared. An SDS aqueous solution obtained by dissolving sodium dodecyl sulfate (SD S) in an ion-exchanged water was prepared, added with the magnetic fine particle dispersion, and subjected to an emulsification treatment for 3 minutes at 50% output using an ultrasonic homogenizer (UP400S manufactured by Hielscher) to prepare a magnetic fine particle emulsion.

After the magnetic fine particle emulsion was added with styrene and divinylbenzene (DVB) and stirred, potassium persulfate (KPS) was added as a polymerization initiator, and a polymerization reaction was performed at 80° C. for 18 hours in an argon gas atmosphere to produce a core 4a. Next, after adding an appropriate amount of magnetic fine particles 4 into a mixture of styrene and DVB and dispersing them, they were mixed with the core 4a together with SDS and KPS and subjected to a polymerization reaction under the same conditions as described above to form an intermediate layer 4b around the core 4a. Moreover, the core 4a provided with the intermediate layer 4b was added with a mixed solution of styrene, DVB, and methacrylic acid together with SDS and KPS, mixed, and subjected to a polymerization reaction under the same conditions to form a polymer layer 6.

### <Measurement of Thickness of Intermediate Layer>

A HAADF-STEM image of the magnetic beads (polymer particles containing magnetic material) 2 was taken at a magnification of 200,000 times so that the number of particles whose entire outline was observed was 10 or more.

For the single magnetic bead 2, a relation between a distance (%) from an outer surface of the magnetic bead 2 to its center 0 and a detection intensity for brightness in a TEM (HAADF) image was determined by the method described with FIG. 1A in the embodiment. The results are shown by Ex. 1 in FIG. 2. From the graph of Ex. 1 in FIG. 2, the thickness of the intermediate layer 4b was determined by the above-mentioned method. Table 1 shows the results.

### <Magnetic Fine Particle Diameter>

100 or more magnetic fine particles 4 whose entire outline was observed were extracted at random from the above-mentioned HAADF-STEM image, and an arithmetic mean of their circle equivalent diameters was determined as a diameter (average) of the magnetic fine particles 4. Table 1 shows the results. The diameter (average) of the magnetic fine particles 4 was 10 nm. The maximum diameter of the magnetic fine particles 4 was 30 nm or less.

### <Diameter of Magnetic Bead>

10 or more magnetic beads 2 whose entire outline was observed were extracted at random from the above-mentioned HAADF-STEM image, and an arithmetic mean of their circle equivalent diameters was determined as a particle diameter (average) of the magnetic beads 2. Table 1 shows the results. The diameter (average) of the magnetic beads 2 was 189 nm. The maximum diameter of the magnetic beads 2 was 1000 nm or less.

### <FT-IR Spectral Analysis of Magnetic Beads>

The magnetic beads 2 were subjected to a FT-IR spectral analysis. The results are shown by Ex. 1 in FIG. 3. In the FT-IR spectral analysis, a sample was applied to a diamond analyzing crystal and subjected to a measurement with a resolution of 4 cm⁻¹ and 32 scans by attenuated total reflection method using a deuterium tri-glycine sulfate (DTGS) detector. In Example 1, as shown by Ex. 1 in FIG. 3, the FT-IR spectrum was confirmed to have a peak in 1620 to 1640 cm⁻¹, and its peak intensity was 1.4 times the peak intensity in 1590 to 1610 cm⁻¹.

### <Modification of Binding Auxiliary Substance to Magnetic Beads>

As an example of the binding auxiliary substance 10c shown in FIG. 5D, streptavidin was added to the magnetic beads 2. In order to modify the surface of the magnetic beads 2 with streptavidin, first, the magnetic beads 2 were dispersed into a phosphate-buffered saline (PBS) adjusted to pH=6.0, and this dispersion was added with N-(3-Dimethylaminopropyl)-N-ethylcarbodiimide hydrochloride and N-hydroxysulfosuccinimide sodium salt and stirred for 30 minutes for reaction. After the reaction, a supernatant was removed, and this reactant was dispersed into a PBS adjusted to pH=7.4, added with streptavidin, and stirred for 3 hours for reaction to synthesize magnetic beads with streptavidin bound to the surface.

### <Producing Sensor Device>

A GMR element was used as a sensor element 32 shown in FIG. 5A used for the sensor unit 25 shown in FIG. 4. A substrate 30 having a carboxyl group (—COOH) was used for the surface of a protection film 30a on the sensor element 32 consisting of the GMR element.

As capture probes 34 formed on the surface of the protection film 30a, a nucleic acid of 5′-AGCTCCTCCTCGGCTGCAAAGACAT-3′—NH2 (Sequence Number: 3) was used.

As the sample solution stored in a sample solution storage 23 shown in FIG. 4, a sample solution containing a single-stranded nucleic acid 10a and a binding auxiliary substance 10b shown in FIG. 5B was used. As the single-stranded nucleic acid, a single-stranded nucleic acid consisting of N1 shown below was used.

### N1:

(Sequence Number: 1)
5′-ATGTCTTTGCAGCCGAGGAGGAGCTGGTGGAGGCTGACGAGGCGGG
CAGTGTGTATGCAGGCATCCTCAGCTACGGGGTGGGCTTCTTCCTGTTC
ATCCTGGTGGT-3′

As the binding auxiliary substance 10b, a biotinylated probe (B1: Biotin-5′-ACCACCAGGATGAACAGGAAGAAGC-3′ (Sequence Number: 5)) shown below was used.

### <Magnetic Bead Solution>

A magnetic bead solution was prepared by mixing the above-mentioned streptavidin-attached magnetic beads 2 with 0.1 mass % of Tween 20 and a phosphate-buffered saline.

### <Measurement of Target Single-Stranded Nucleic Acid in Sample Solution>

The measurement of the target single-stranded nucleic acid in the sample solution was performed in the following procedure.

(1) A sample solution was prepared.
(2) A mixed solution obtained in (1) was heated at 97° C. for 20 minutes.
(3) After cooling the heated solution with ice, it was immediately injected into the sample solution storage 23 of the nucleic acid detection cartridge 20 shown in FIG. 4, set in a nucleic acid detector, and reached onto the sensor element 23 of the sensor unit 25.
(4) The solution was allowed to stand still for 30 minutes while being reached on the sensor element 23.
(5) Next, a washing liquid stored in the washing liquid storage 24 of the nucleic acid detection cartridge 20 was allowed to reach onto the sensor element 32 and wash the surface of the sensor element 32.
(6) An external magnetic field of 30 Oe was applied in the in-plane direction of the sensor element 32, and the measurement of the resistance value obtained by converting the output value of the sensor element 32 was started.
(7) While continuing to measure the resistance value of the sensor element 32, the magnetic bead solution stored in the magnetic bead solution storage 22 of the nucleic acid detection cartridge 20 was transmitted onto the sensor element 32.
(8) A resistance change rate (% output) of the sensor element 32 for 20 minutes after the magnetic bead solution was transferred was measured.

FIG. 6A illustrates an example of the measurement. Table 1 shows the measurement results of the resistance change rate r1₂₀ in 20 minutes. Table 1 also shows the measurement results of the resistance change rate r2₂₀ in 20 minutes after measuring the background signal (noise signal) using another sensor element (not illustrated). The value of r2₂₀/r1₂₀ in Table 1 represents a ratio of the magnitude of the noise signal to the required detection signal and is preferably lower. As shown in FIG. 5E, the value of the resistance change rate r1₂₀ corresponds to the number of magnetic beads 2 indirectly bound to the single-stranded nucleic acid 10a as a target substance captured by the capture probes 34 and is preferably larger. This is because detection accuracy is improved.

### Example 2

The magnetic beads 2 were manufactured in the same manner as in Example 1, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Example 1 as shown in Table 1. Table 1 shows the results.

### Example 3

The magnetic beads 2 were manufactured in the same manner as in Example 2, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was further smaller than that in Example 2 as shown in Table 1. Table 1 shows the results.

### Comparative Example 1

The magnetic beads 2 were manufactured in the same manner as in Example 3, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was further smaller than that in Example 3 as shown in Table 1. Table 1 shows the results. Moreover, a relation between a distance from the outer surface to the center of the magnetic bead and a concentration (detection intensity of image brightness) of the magnetic fine particles according to Comparative Example 1 is shown by Cex. 1 in FIG. 2.

### Example 4

The magnetic beads 2 were manufactured in the same manner as in Example 1, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 1 as shown in Table 1. Table 1 shows the results.

### Example 5

The magnetic beads 2 were manufactured in the same manner as in Example 4, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was further larger than that in Example 4 as shown in Table 1. Table 1 shows the results. Moreover, a relation between a distance (%) from the outer surface to the center of the magnetic bead 2 and a detection intensity for brightness in a TEM (HAADF) image was obtained. The results are shown by Ex. 5 in FIG. 2.

### Example 6

The magnetic beads 2 were manufactured in the same manner as in Example 5, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was further larger than that in Example 5 as shown in Table 1. Table 1 shows the results.

### Comparative Example 2

The magnetic beads 2 were manufactured in the same manner as in Example 6, and the same measurements and evaluations as in Example 6 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was further larger than that in Example 6 as shown in Table 1. Table 1 shows the results. Moreover, a relation between a distance from the outer surface to the center of the magnetic bead and a concentration (detection intensity of image brightness) of the magnetic fine particles according to Comparative Example 2 is shown by Cex. 2 in FIG. 2. Moreover, FIG. 6B shows an example of the output of the sensor element 32 according to Comparative Example 2.

### Example 7

The magnetic beads 2 were manufactured in the same manner as in Example 1, and the same measurements and evaluations as in Example 1 were performed, except for changing the solvent to trioctylamine in the manufacturing conditions of the magnetic fine particles 4 and performing a thermal decomposition at 367° C. for 2 hours so that the average diameter of the magnetic fine particles was larger than that in Example 1 as shown in Table 2. Table 2 shows the results.

### Example 8

The magnetic beads 2 were manufactured in the same manner as in Example 7, and the same measurements and evaluations as in Example 1 were performed, except for setting the thermal decomposition time to 6 hours in the manufacturing conditions of the magnetic fine particles 4 so that the average diameter of the magnetic fine particles was further larger than that in Example 7 as shown in Table 2. Table 2 shows the results.

### Comparative Example 3

The magnetic beads 2 were manufactured in the same manner as in Example 8, and the same measurements and evaluations as in Example 1 were performed, except for setting the thermal decomposition time to 12 hours in the manufacturing conditions of the magnetic fine particles 4 so that the average diameter of the magnetic fine particles was further larger than that in Example 8 as shown in Table 2. Table 2 shows the results.

### Example 9

The magnetic beads 2 were manufactured in the same manner as in Example 1, and the same measurements and evaluations as in Example 1 were performed, except for changing the solvent to hexadecane in the manufacturing conditions of the magnetic fine particles 4 and performing a thermal decomposition at 280° C. for 2 hours so that the average diameter of the magnetic fine particles 4 was smaller than that in Example 1 as shown in Table 2. Table 2 shows the results.

### Comparative Example 4

The magnetic beads 2 were manufactured in the same manner as in Example 9, and the same measurements and evaluations as in Example 1 were performed, except for performing a thermal decomposition at 265° C. for 2 hours in the manufacturing conditions of the magnetic fine particles 4 so that the average diameter of the magnetic fine particles 4 was further smaller than that in Example 9 as shown in Table 2. Table 2 shows the results.

### Comparative Example 5

The magnetic beads 2 were manufactured in the same manner as in Comparative Example 4, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Comparative Example 4 as shown in Table 2. Table 2 shows the results.

### Comparative Example 6

The magnetic beads 2 were manufactured in the same manner as in Comparative Example 3, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Comparative Example 3 as shown in Table 2. Table 2 shows the results.

### Comparative Example 7

The magnetic beads 2 were manufactured in the same manner as in Comparative Example 4, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Comparative Example 4 as shown in Table 2. Table 2 shows the results.

### Comparative Example 8

The magnetic beads 2 were manufactured in the same manner as in Comparative Example 3, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Comparative Example 3 as shown in Table 2. Table 2 shows the results.

### Example 10

The magnetic beads 2 were manufactured in the same manner as in Example 7, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Example 7 as shown in Table 2. Table 2 shows the results.

### Example 11

The magnetic beads 2 were manufactured in the same manner as in Example 8, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Example 8 as shown in Table 2. Table 2 shows the results.

### Example 12

The magnetic beads 2 were manufactured in the same manner as in Example 9, and the same measurements and evaluations as in Example 1 were performed, except for increasing the number of magnetic fine particles 4 contained in the core 4a and decreasing the amounts of magnetic fine particles, styrene, and DVB added at the time of producing the intermediate layer 4b so that the intermediate layer thickness (%) was smaller than that in Example 9 as shown in Table 2. Table 2 shows the results.

### Example 13

The magnetic beads 2 were manufactured in the same manner as in Example 7, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 7 as shown in Table 2. Table 2 shows the results.

### Example 14

The magnetic beads 2 were manufactured in the same manner as in Example 8, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 8 as shown in Table 2. Table 2 shows the results.

### Example 15

The magnetic beads 2 were manufactured in the same manner as in Example 9, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 9 as shown in Table 2. Table 2 shows the results.

### Example 16

The magnetic beads 2 were manufactured in the same manner as in Example 9, and the same measurements and evaluations as in Example 1 were performed, except for weakening the ultrasonic output and shortening the irradiation time at the time of an emulsification treatment in the manufacturing conditions of the magnetic beads 2 so that the average diameter ratio between magnetic fine particles and polymer magnetic particles was smaller than that in Example 9 as shown in Table 3. Table 3 shows the results.

### Examples 17-23

The magnetic beads 2 were manufactured in the same manner as in Example 16, and the same measurements and evaluations as in Example 23 were performed, except for sequentially weakening the ultrasonic output and sequentially lengthening the irradiation time at the time of an emulsification treatment in the manufacturing conditions of the magnetic beads 2 so that the average diameter ratio between magnetic fine particles and polymer magnetic particles was sequentially increased compared to Example 16 as shown in Table 3. Table 3 shows the results.

### Example 24

The magnetic beads 2 were manufactured in the same manner as in Example 11, and the same measurements and evaluations as in Example 1 were performed, except for lengthening the ultrasonic irradiation time at the time of an emulsification treatment in the manufacturing conditions of the magnetic beads 2 so that the average diameter ratio between magnetic fine particles and polymer magnetic particles was larger than that in Example 11 as shown in Table 3. Table 3 shows the results.

### Example 25

The magnetic beads 2 were manufactured in the same manner as in Example 8, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 24 as shown in Table 3. Table 3 shows the results.

### Example 26

The magnetic beads 2 were manufactured in the same manner as in Example 11, and the same measurements and evaluations as in Example 1 were performed, except for weakening the ultrasonic irradiation output and lengthening the ultrasonic irradiation time at the time of an emulsification treatment in the manufacturing conditions of the magnetic beads 2 so that the average diameter ratio between magnetic fine particles and polymer magnetic particles was smaller than that in Example 12 as shown in Table 3. Table 3 shows the results.

### Example 27

The magnetic beads 2 were manufactured in the same manner as in Example 26, and the same measurements and evaluations as in Example 1 were performed, except for decreasing the number of magnetic fine particles 4 contained in the core 4a and increasing the amounts of magnetic fine particles, styrene, and DVB added at the time of producing the intermediate layer 4b so that the intermediate layer thickness (%) was larger than that in Example 26 as shown in Table 3. Table 3 shows the results.

### Example 32

The magnetic beads 2 were manufactured in the same manner as in Example 1, and the same measurements and evaluations as in Example 1 were performed, except for increasing the amount of polymerization initiator in the manufacturing conditions of the magnetic beads 2 so that an intensity ratio of a peak in 1620 to 1640 cm⁻¹ to a peak in 1590 to 1610 cm⁻¹ in a FT-IR measurement was small as shown in Table 4. Table 4 shows the results.

### Example 33

The magnetic beads 2 were manufactured in the same manner as in Example 32, and the same measurements and evaluations as in Example 1 were performed, except for increasing the amount of polymerization initiator in the manufacturing conditions of the magnetic beads 2 so that an intensity ratio of a peak in 1620 to 1640 cm⁻¹ to a peak in 1590 to 1610 cm⁻¹ in a FT-IR measurement was further smaller than that in Example 32 as shown in Table 4. Table 4 shows the results. The results of the FT-IR spectrum analysis for the magnetic beads 2 according to Example 33 are shown by Ex. 33 in FIG. 3.

### Example 34

The magnetic beads 2 were manufactured in the same manner as in Example 1, and the same measurements and evaluations as in Example 1 were performed, except for decreasing the amount of polymerization initiator in the manufacturing conditions of the magnetic beads 2 so that an intensity ratio of a peak in 1620 to 1640 cm⁻¹ to a peak in 1590 to 1610 cm⁻¹ in a FT-IR measurement was larger than that in Example 1 as shown in Table 4. Table 4 shows the results.

### <Evaluation>

In particular, as shown in Table 1 and Table 2, it was confirmed that the magnetic beads of each example having the diameter of the magnetic fine particles and the thickness of the intermediate layer within the predetermined ranges has a large signal intensity r1₂₀ and a small signal intensity r2₂₀ (noise component) and is favorably used as a part of a sensor medium for detecting, for example, a double-stranded nucleic acid or a single-stranded nucleic acid 10a. Note that, the signal intensity r1₂₀ is preferably 0.5 or more and is more preferably 1.5 or more, and a noise ratio r2₂₀/r1₂₀ is preferably 0.5 or less and is more preferably 0.2 or less.

It was confirmed that the magnetic beads of Comparative Example 1 (the thickness of the intermediate layer was small) has a large signal intensity r2₂₀ (noise component) and also has a large noise ratio r2₂₀/r1₂₀. The reason for this is thought to be that, in Comparative Example 1, the saturation magnetization increased, the specific gravity of polymer particles increased, and the sedimentation velocity increased. Moreover, the magnetic beads of Comparative Example 2 (the thickness of the intermediate layer was large) had an insufficient signal intensity r1₂₀. This is probably because the saturation magnetization of the magnetic beads decreased.

As shown in Table 2, it was confirmed that when the magnetic fine particles have an average diameter of 5 nm or more and 30 nm or less, the signal intensity r1₂₀ is large, and the signal intensity r2₂₀ (noise component) is small. Note that, when the magnetic fine particles have an average diameter of larger than 30 nm (Comparative Example 3), the noise ratio deteriorates. This is probably because the magnetic beads magnetically aggregated and settled, and the noise signal thus increased. Moreover, when the magnetic fine particles had an average diameter of smaller than 5 nm (Comparative Example 4), the signal intensity r1₂₀ was small. This is probably because the proportion of surface components not contributing to the saturation magnetization of the magnetic fine particles was relatively large, and the saturation magnetization decreased.

As shown in Table 3, it was confirmed that the magnetic beads with good characteristics can be produced when the diameter ratio of the magnetic fine particles to the magnetic beads was 0.005 or more and 0.25 or less. As shown in Table 4, it was also confirmed that the noise ratio r2₂₀/r1₂₀ is small when the intensity ratio of the peak in 1620 to 1640 cm⁻¹ to the peak in 1590 to 1610 cm⁻¹ in the FT-IR spectrum analysis is 0.2 or more and 3.0 or less, and that the noise ratio r2₂₀/r1₂₀ was smaller when the intensity ratio of the peak in 1620 to 1640 cm⁻¹ to the peak in 1590 to 1610 cm⁻¹ in the FT-IR spectrum analysis was 0.5 or more and 3.0 or less.

TABLE 1

| | | Average | | Average | | | | |
|---|---|---|---|---|---|---|---|---|
| | | Diameter of | Average | Diameter Ratio | | | | |
| | Intermediate | Magnetic | Diameter of | of Magnetic | | | | |
| | Layer | Fine | Magnetic | Fine Particles | Saturation | | | |
| | Thickness | Particles | Beads | to Magnetic | Magnetization | r1₂₀ | r2₂₀ | |
| | [%] | [nm] | [nm] | Beads | [emu/g] | [%] | [%] | r2₂₀/r1₂₀ |
| Comp. | 3.4 | 11 | 198 | 0.06 | 52 | 5.6 | 4.9 | 0.88 |
| Ex. 1 | | | | | | | | |
| Ex. 3 | 5.0 | 11 | 199 | 0.06 | 51 | 5.3 | 2.5 | 0.47 |
| Ex. 2 | 10.5 | 11 | 195 | 0.06 | 48 | 5.2 | 0.9 | 0.17 |
| Ex. 1 | 16.5 | 11 | 189 | 0.06 | 43 | 4.8 | 0.1 | 0.02 |
| Ex. 4 | 25.2 | 11 | 201 | 0.05 | 28 | 3.0 | 0.1 | 0.03 |
| Ex. 5 | 41.5 | 11 | 198 | 0.06 | 18 | 1.7 | 0.03 | 0.02 |
| Ex. 6 | 60.0 | 11 | 202 | 0.05 | 10 | 0.6 | <0.01 | <0.01 |
| Comp. | 75.0 | 11 | 200 | 0.06 | 5 | 0.1 | <0.01 | <0.01 |
| Ex. 2 | | | | | | | | |

TABLE 2

| | | Average | | Average | | | | |
|---|---|---|---|---|---|---|---|---|
| | | Diameter of | Average | Diameter Ratio | | | | |
| | Intermediate | Magnetic | Diameter of | of Magnetic | | | | |
| | Layer | Fine | Magnetic | Fine Particles | Saturation | | | |
| | Thickness | Particles | Beads | to Magnetic | Magnetization | r1₂₀ | r2₂₀ | |
| | [%] | [nm] | [nm] | Beads | [emu/g] | [%] | [%] | r2₂₀/r1₂₀ |
| Ex. 1 | 16.5 | 11 | 189 | 0.06 | 43 | 4.8 | 0.1 | 0.02 |
| Ex. 7 | 15.9 | 26 | 188 | 0.14 | 49 | 5.2 | 0.7 | 0.13 |
| Ex. 8 | 15.5 | 30 | 191 | 0.16 | 51 | 5.4 | 2.5 | 0.46 |
| Comp. | 15.5 | 40 | 191 | 0.21 | 53 | 5.6 | 5.3 | 0.95 |
| Ex. 3 | | | | | | | | |
| Comp. | 16.0 | 3.8 | 190 | 0.02 | 6 | 0.2 | 0.01 | 0.05 |
| Ex. 4 | | | | | | | | |
| Ex. 9 | 16.1 | 5 | 189 | 0.03 | 19 | 1.7 | 0.01 | 0.01 |
| Comp. | 10.1 | 3.8 | 190 | 0.02 | 10 | 1.4 | 0.8 | 0.57 |
| Ex. 5 | | | | | | | | |
| Comp. | 10.3 | 40 | 195 | 0.21 | 55 | 5.8 | 5.5 | 0.95 |
| Ex. 6 | | | | | | | | |
| Ex. 2 | 10.5 | 11 | 195 | 0.06 | 48 | 5.2 | 0.9 | 0.17 |
| Ex. 10 | 10.5 | 26 | 189 | 0.14 | 50 | 5.3 | 1.0 | 0.19 |
| Ex. 11 | 10.7 | 30 | 195 | 0.15 | 52 | 5.5 | 1.1 | 0.20 |
| Ex. 12 | 10.6 | 5 | 192 | 0.03 | 20 | 1.8 | 0.50 | 0.28 |
| Comp. | 40.6 | 3.8 | 210 | 0.02 | 4 | 0.1 | <0.01 | <0.01 |
| Ex. 7 | | | | | | | | |
| Comp. | 40.0 | 40 | 200 | 0.20 | 48 | 2.5 | 2.8 | 1.12 |
| Ex. 8 | | | | | | | | |
| Ex. 5 | 41.5 | 11 | 198 | 0.06 | 33 | 1.7 | 0.03 | 0.02 |
| Ex. 13 | 40.5 | 26 | 188 | 0.14 | 42 | 2.0 | 0.04 | 0.02 |
| Ex. 14 | 41.2 | 30 | 189 | 0.16 | 46 | 2.0 | 0.10 | 0.05 |
| Ex. 15 | 40.8 | 5 | 190 | 0.03 | 17 | 1.5 | <0.01 | <0.01 |

TABLE 3

| | | Average | | Average | | | | |
|---|---|---|---|---|---|---|---|---|
| | | Diameter of | Average | Diameter Ratio | | | | |
| | Intermediate | Magnetic | Diameter of | of Magnetic | | | | |
| | Layer | Fine | Magnetic | Fine Particles | Saturation | | | |
| | Thickness | Particles | Beads | to Magnetic | Magnetization | r1₂₀ | r2₂₀ | |
| | [%] | [nm] | [nm] | Beads | [emu/g] | [%] | [%] | r2₂₀/r1₂₀ |
| Ex. 16 | 15.8 | 5 | 1000 | 0.005 | 25 | 2.5 | 0.62 | 0.25 |
| Ex. 17 | 16.0 | 5 | 495 | 0.01 | 23 | 2.2 | 0.44 | 0.20 |
| Ex. 18 | 15.8 | 5 | 305 | 0.02 | 23 | 2.4 | 0.25 | 0.10 |
| Ex. 19 | 15.5 | 5 | 210 | 0.02 | 21 | 2.3 | 0.22 | 0.10 |
| Ex. 20 | 15.5 | 5 | 99 | 0.05 | 20 | 2.2 | 0.12 | 0.05 |
| Ex. 21 | 16.1 | 5 | 51 | 0.10 | 19 | 2.1 | 0.12 | 0.06 |
| Ex. 22 | 15.6 | 5 | 30 | 0.17 | 19 | 2.1 | 0.11 | 0.05 |
| Ex. 23 | 15.9 | 5 | 21 | 0.24 | 18 | 2.0 | 0.25 | 0.13 |
| Ex. 12 | 10.6 | 5 | 192 | 0.03 | 20 | 1.8 | 0.50 | 0.28 |
| Ex. 2 | 10.5 | 11 | 195 | 0.06 | 48 | 5.2 | 0.90 | 0.17 |
| Ex. 11 | 10.7 | 30 | 195 | 0.15 | 52 | 5.5 | 1.1 | 0.20 |
| Ex. 15 | 40.8 | 5 | 190 | 0.03 | 17 | 1.5 | <0.01 | <0.01 |
| Ex. 5 | 41.5 | 11 | 198 | 0.06 | 18 | 1.7 | 0.03 | 0.02 |
| Ex. 13 | 40.5 | 26 | 188 | 0.14 | 48 | 1.8 | 0.04 | 0.02 |
| Ex. 24 | 10.7 | 30 | 120 | 0.25 | 52 | 5.5 | 0.6 | 0.11 |
| Ex. 25 | 41.2 | 30 | 121 | 0.25 | 31 | 3.0 | 0.10 | 0.03 |
| Ex. 26 | 10.6 | 5 | 890 | 0.006 | 20 | 1.8 | 0.44 | 0.24 |
| Ex. 27 | 40.8 | 5 | 998 | 0.005 | 21 | 0.8 | 0.10 | 0.13 |

TABLE 4

| | | Average | | Average | | | | | |
|---|---|---|---|---|---|---|---|---|---|
| | | Diameter of | Average | Diameter Ratio | | | | | |
| | Intermediate | Magnetic | Diameter of | of Magnetic | | FT-IR | | | |
| | Layer | Fine | Magnetic | Fine Particles | Saturation | Peak | | | |
| | Thickness | Particles | Beads | to Magnetic | Magnetization | Intensity | r1₂₀ | r2₂₀ | |
| | [%] | [nm] | [nm] | Beads | [emu/g] | Ratio | [%] | [%] | r2₂₀/r1₂₀ |
| Ex. 1 | 16.5 | 11 | 189 | 0.06 | 43 | 1.4 | 4.8 | 0.1 | 0.02 |
| Ex. 32 | 16.2 | 11 | 188 | 0.06 | 43 | 0.5 | 4.8 | 0.9 | 0.19 |
| Ex. 33 | 16.0 | 11 | 190 | 0.06 | 44 | 0.2 | 4.8 | 2.1 | 0.44 |
| Ex. 34 | 16.4 | 11 | 192 | 0.06 | 44 | 3.0 | 4.8 | 0.08 | 0.02 |

Description of the Reference Numerical
2 . . . magnetic bead (polymer particle containing magnetic material)
4 . . . magnetic fine particle
4a . . . core
4b . . . intermediate layer
6 . . . polymer layer
10a . . . single-stranded nucleic acid
10c . . . binding auxiliary substance
20 . . . nucleic acid detection cartridge (sensor device)
22 . . . magnetic bead storage
23 . . . sample solution storage
24 . . . washing liquid storage
25 . . . sensor unit
26 . . . waste liquid storage
27 . . . connection section
28 . . . magnetic field application unit
30 . . . substrate
32 . . . sensor element
34 . . . capture probe

What is claimed is:

1. A polymer particle containing magnetic material, comprising:
a core including magnetic fine particles having an average diameter of 5 nm or more and 30 nm or less;
an intermediate layer located outside the core and having a lower concentration of the magnetic fine particles than the core; and
a polymer layer covering the intermediate layer,
wherein a thickness of the intermediate layer is 5% or more and 60% or less of a radius of the polymer particle containing magnetic material.

2. The polymer particle containing magnetic material according to claim 1, wherein a thickness of the intermediate layer is 10% or more and 42% or less of a radius of the polymer particle containing magnetic material.

3. The polymer particle containing magnetic material according to claim 1, wherein a ratio (x1/x2) of a diameter (x1) of the magnetic fine particles to a diameter (x2) of the polymer particle containing magnetic material is 0.005 or more and 0.25 or less.

4. The polymer particle containing magnetic material according to claim 1, wherein a polymer constituting the polymer layer contains an unpolymerized vinyl group.

5. The polymer particle containing magnetic material according to claim 1, wherein an intensity ratio of a peak in 1620 to 1640 cm⁻¹ to a peak in 1590 to 1610 cm⁻¹ in a FT-IR spectrum is 0.2 or more and 3.0 or less.

6. The polymer particle containing magnetic material according to claim 1, further comprising a portion capable of directly or indirectly binding with a target substance.

7. A medium for sensors comprising the polymer particle containing magnetic material according to claim 1.

8. A sensor device comprising a sensor unit for detecting a magnetism of the polymer particle containing magnetic material according to claim 1 binding with a target substance.
